(19) United States
(12) Patent Application Publication
(10) Pub. No.: US 2021/0369674 A1
(43) Pub. Date: Dec. 2, 2021

(54) HEALTH MANAGEMENT COMPOSITIONS AND METHODS OF MAKING AND USING THEREOF

(71) Applicant: Seattle Gummy Company, Seattle, WA (US)

(72) Inventor: Feng Wan, Issaquah, WA (US)

(21) Appl. No.: 16/322,497

(22) PCT Filed: Aug. 3, 2017
(86) PCT No.: PCT/US17/45386

§ 371 Date: Feb. 1, 2019

Related U.S. Application Data
(60) Provisional application No. 62/371,136, filed on Aug. 4, 2016.

(51) Int. Cl. A61K 31/4045; A23L 33/125; A23L 33/175; A23L 33/16; A23L 33/105; A23L 33/12; A23L 33/155; A61K 31/205; A61K 31/385; A61K 36/258; A61K 31/616; A61K 31/12; A61K 36/9066; A61K 36/88; A61K 36/9068; A61K 36/28; A61K 36/484; A61K 31/165; A61K 36/52; A61K 36/49; A61K 36/87; A61K 31/122; A61K 31/047; A61K 36/45; A61K 31/01; A61K 36/815; A61K 36/481; A61K 36/16; A61K 35/10; A61K 31/706; A61K 31/7004; A61K 31/07; A61K 31/375; A61K 31/593; A61K 31/355; A61K 31/51; A61K 31/525; A61K 31/4415; A61K 31/714; A61K 31/519; A61K 31/197; A61K 47/42; A61K 47/36; A61K 9/06; A61K 38/45; A61K 33/06; A61K 9/00

(52) U.S. Cl. CPC A61K 31/4045; A23V 2002/00; A23L 33/175; A23L 33/16; A23L 33/105; A23L 33/12; A23L 33/155; A61K 31/205; A61K 31/385; A61K 36/258; A61K 31/616; A61K 31/12; A61K 36/9066; A61K 36/88; A61K 36/9068; A61K 36/28; A61K 36/484; A61K 31/165; A61K 36/52; A61K 36/49; A61K 36/87; A61K 31/122; A61K 31/047; A61K 36/45; A61K 31/01; A61K 36/815; A61K 36/481; A61K 36/16; A61K 35/10; A61K 31/706; A61K 31/7004; A61K 31/07; A61K 31/375; A61K 31/593; A61K 31/355; A61K 31/51; A61K 31/525; A61K 31/4415; A61K 31/714; A61K 31/519; A61K 31/197; A61K 47/42; A61K 47/36; A61K 9/06; A61K 38/45; A61K 33/06; A61K 9/0056; A23L 33/125

(57) ABSTRACT

The nutraceutical composition includes a neuroprotecting composition, an anti-inflammatory composition, a cardiovascular protecting composition, an antioxidant composition, and a gelling component in a sufficient amount to provide a cohesive gelled product.

### CROSS-REFERENCES TO RELATED APPLICATIONS

This application claims the benefit of priority from, and hereby incorporates by reference the entire disclosure, co-pending U.S. Provisional Application for Patent Ser. No. 62/371,136, filed Aug. 4, 2016.

### TECHNICAL FIELD

The application relates generally to nutraceutical composition in gelled or gummy formula, methods of administration of various gummy or gelled compositions for nutraceuticals, and kits comprising various gummy compositions for nutraceuticals.

### BACKGROUND

Aging degrades motivation, cognition, sensory modalities and physical capacities, essentially dimming zestful living. Bradykinesis (declining physical movement) is a highly reliable biomarker for aging and mortality risk. For aging humans, maintaining zestful health and performance into later years may provide greater social and economic benefits than simply prolonging lifespan.

Key factors implicated in aging include reactive oxygen species, inflammatory processes, insulin resistance, and mitochondrial dysfunction. Oxidative stress arises from diverse causes such as xenobiotics, nicotinamide adenine dinucleotide phosphate (NADPH) oxidases, hypoxia, aging, disease process, mitochondrial function and exposure to ionizing radiations. Free radicals damage lipids, proteins, DNA and cellular structures. Oxidative DNA base damage, chromosome aberrations, genomic instability and telomere alterations are among the most deleterious consequences of oxidative processes consequently making high doses potentially mutagenic.

Several studies investigating various indicators of DNA damage, including oxidative base damage, telomere shortening, chromosome fragments, chromosome loss and aneuploidy have consistently shown that DNA damage increases proportionately with age and are considered valid markers of ageing. Genomic aberration increases the risk of carcinogenesis and may actually accelerate the ageing process by contributing to cell dysfunction and death. Chronic oxidative stress such as that associated with ageing is a significant contributor to the accumulation of damaged DNA and chromosome rearrangements. The processes associated with ageing are the most common causes of chronic oxidative stress; however, many disease states including neurodegenerative diseases, cancer, progerias, atherosclerosis, autoimmunity and diabetes include chronic oxidative stress as a significant factor in the progression of the disease. The age-related increase in DNA damage is also associated with progressive mitochondrial dysfunction and rising free radical production, which may contribute to progressive physiological dysfunction, deterioration and mortality. Cellular responses mediating defense, repair and replacement also appear to attenuate with age and contribute to ageing.

### SUMMARY

The application provides nutraceutical compositions and kits for maintaining good health and anti-aging management and methods of making and using thereof.

In one aspect, the application provides a nutraceutical composition. In one embodiment, the nutraceutical composition includes a neuroprotecting composition, an anti-inflammatory composition, a cardiovascular protecting composition, an antioxidant composition, and a gelling composition in a sufficient amount to provide a cohesive gelled product.

In one embodiment, the nutraceutical composition further includes a telomerase activating composition. In one embodiment, the nutraceutical composition further includes a mitochondria boosting composition. In one embodiment, the nutraceutical composition further includes a vitamin composition. In one embodiment, the nutraceutical composition further includes a mineral composition.

In one embodiment, the neuroprotecting composition comprises melatonin, L-carnitine, acetyl L-carnitine, alpha-lipoic acid, ginseng, Allium sativum, Bacopa monnierae, Centella asiatica, Celastrus paniculatus, Nicotiana tabaccum, Withania somnifera, Ricinus communis, Salvia officinalis, Ginkgo biloba, Huperiza serrata, Angelica sinensis, Uncaria tomentosa, Hypericum perforatum, Physostigma venosum, Acorns calmus, Curcuma longa, Terminalia chebula, Crocus sativus, Enhydra fluctuans, Valeriana wallichii, Glycyrrhiza glabra, Ledebouriella divaricata, Scutellaria baicalensis, Angelica pubescens, Morus alba, Salvia miltiorrhiza, Uncaria rhynchophylla, Ligusticum chuanxiong, Scutellaria baicalensis, curcumin, Gastrodia elata, Cistanche deserticola, Cistanche tubulosa, Paeoniae alba Radix, Polygalaw radix (PRE), Polygala tenuifolia, Pueraria lobata, Alpiniaw Oxyphyllae, Fructus (AOE), Polygonum cuspidatum, its extract, powder, isolates, or derivatives thereof. In one embodiment, the ginseng comprises Asian ginseng (Panax ginseng), Siberian ginseng (Eleutherococcus senticosus), American ginseng (Panax quinquefolius), Indian ginseng (Withania somnifera), vitamin E, or a combination thereof.

In one embodiment, the anti-inflammatory composition comprises acetylsalicylic acid, bioflavonoids, omega-3 fatty acids, white willow, curcumin, turmeric, saffron, ginger, Boswellai serrate, chamomile, licorice, arnica, witch hazel, Echinacea, bromelain from pineapple, yarrow, oak bark, walnut leaf, fenugreek, aloe vera, calendula, capsaicin, mallow, wild pansy, its extract, powder, or isolates thereof.

In one embodiment, the cardiovascular protecting composition comprises omega-3 fatty acids, cod liver oil, fish oil, algae oil, flax seed oil, or a combination thereof.

In one embodiment, the antioxidant composition comprises bioflavonoids, pomegranate, Ginkgo biloba, green tea, garlic, grapeseed, vitamin E, vitamin C, resveratrol, coenzyme Q10, quercetin, rutin, lycopene, L-glutathione, N-acetyl cysteine, phenolics, anthocyanins, flavonoids, anthracenes, carotenoids, zeaxanthin, blackberry, elderberry, cranberry, blueberry, saffron, Sangre de grado (dragon's blood), Lyceum barbarum (Gouqizi), its extract, power, or isolates thereof.

In one embodiment, the telomerase activating composition is capable of stimulating telomerase activity. In one embodiment, the telomerase activating composition comprises Astragalus, its extract or isolates thereof, astragaloside, astragenol, cycloastragenol, or derivatives thereof.

In one embodiment, the mitochondria boosting composition is capable of protecting or boosting mitochondria activity. In one embodiment, the mitochondria boosting composition comprises acetyl L-carnitine, alpha-lipoic acid, coenzyme Q10 (CoQ10, or ubiquinone), vitamin B, vitamin D, omega-3 fatty acids, magnesium, D-ribose, or a derivative or combination thereof. In one embodiment, the vitamin B comprises Vitamin B12, Vitamin B3, riboflavin, thiamine, and B6, or a combination thereof. In one embodiment, the mitochondria composition comprises nicotinamide riboside (NR), which is a naturally occurring metabolite of Vitamin B3 (niacin). In one embodiment, the mitochondria boosting composition comprises Q10 and Shilajit extract or powder thereof.

The composition may further include a vitamin composition, a mineral composition, a stimulant composition, an amino acid composition.

In one embodiment, the vitamin composition comprises a vitamin A composition, a vitamin B composition, vitamin C, vitamin D, vitamin E, or a combination thereof. In one embodiment, the vitamin A composition comprises beta-carotene, lutein, or a combination thereof. In one embodiment, the Vitamin B composition comprises Vitamin B1 (thiamine), niacin (B3), vitamin B6, vitamin B12, folic acid, or derivatives thereof. In one embodiment, the vitamin composition comprises Vitamin B1 (thiamine), Vitamin B2 (riboflavin), niacin, vitamin B6, vitamin B12, folic acid, pantothenic acid, biotin, or derivatives thereof. In one embodiment, the vitamin B composition comprises Vitamin B1 (thiamine) not less than 1.2 mg, Vitamin B2 (riboflavin) not less than 1.3 mg, niacin not less than 16 mg, vitamin B6 not less than 1.5 mg, vitamin B12 not less than 2.4 mcg, folate not less than 400 mcg, pantothenic acid not less than 5 mg, biotin not less than 30 mcg. In one embodiment, the vitamin composition comprises Vitamin E not less 60 IU, Vitamin C not less than 90 mg, or beta-carotene not less than 2000 IU. In one embodiment, Vitamin D is not less than 600 IU.

In one embodiment, the mineral composition comprises salts or chelates of chromium pocolinate, magnesium, selenium, selenium yeast, potassium, or a combination thereof. In one embodiment, the mineral composition further comprises calcium, iron, zinc, sodium, chloride, potassium, copper, molybdenum, manganese, phosphorus, iodine, nickel, or a combination thereof. In one embodiment, the chelates are amino acid chelates.

In one embodiment, the nutraceutical composition further comprises an amino acid composition. In one embodiment, the amino acid composition comprises at least one essential amino acid. In one embodiment, the amino acid composition comprises phenylalanine, valine, threonine, tryptophan, methionine, leucine, isoleucine, lysine, and histidine. In one embodiment, the amino acid composition comprises leucine, isoleucine, and valine.

In one embodiment, the gelling composition comprises gelatin, starch, pectin, gellan gum, gum Arabic, carrageenans, guar, agar, alginate, locust bean gum, xanthan, or derivatives thereof. Pectin may include apple pectin, citrus pectin, or a combination thereof. In one embodiment, the gelling composition comprises donkey hide gelatin, beef gelatin or port gelatin. In one embodiment, the starch comprises corn starch, tapioca starch, potato starch, wheat starch, dextrins, maltodextrins, thin-boiling starch, high amylose corn starch, instant starches, or derivatives thereof.

In one embodiment, the nutraceutical composition may include pectin about 1% to about 20% by weight based on the total weight of the composition. In one embodiment, the nutraceutical composition may include gelatin about 1% to about 20% by weight based on the total weight of the composition. In one embodiment, the nutraceutical composition may include pectin about 1% to about 20% by weight and hydrolyzed collagen about 1% to about 10% by weight.

In one embodiment, the composition is essentially free of gelatin. In one embodiment, the gelling composition consists essentially of pectin and gelatin. In one embodiment, the gelling composition consists essentially of pectin and hydrolyzed collagen in a ratio from about 10:1 to about 1:1. In one embodiment, the nutraceutical composition may include about 0.25% to about 0.75% by weight gellan gum and about 2% to about 3% by weight carrageenan based on the total weight of the composition. In one embodiment, the nutraceutical composition may include about 0.25% to about 0.5% by weight gellan gum and about 2.5% to about 3% by weight carrageenan based on the total weight of the composition.

In one embodiment, the nutraceutical composition may further comprise an additive. In one embodiment, the additive may include sweeteners, food acids, flavoring agents, coloring agents, humectants, bulking agents, fatty acids, triglycerides, emulsifiers, thickeners, or and a mixture thereof.

In one embodiment, the sweetener may be sugar, syrup, glucose syrup, corn syrup, high fructose corn syrup, sugar cane syrup, tapioca syrup, juice concentrate, or mixtures thereof. In one embodiment, the sweetener may include sugar, glucose syrup, corn syrup, high fructose corn syrup, juice concentrate, tapioca syrup, agave syrup, brown rice syrup, high maltose syrup, invert sugar, artificial sweeteners, licorice or its extracts or isolates, or a mixture thereof. In one embodiment, the sweetener comprises essentially of licorice or its extracts or isolates thereof. In one embodiment, the sweetener may include a low glycemic index sugar. The low glycemic index sugar may be a sugar having a glycemic index less than 10, 20, 30, 40, 50, 60 or 70.

In one embodiment, the sweetener may include rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-phenylal-anine 1-methyl ester, salts thereof, and combinations thereof.

In one embodiment, the sweetener may be an artificial sweetener. In one embodiment, the artificial sweetener may include saccharin, saccharin salts, cyclamic acid, cyclamic acid salts, aspartame, sucralose, acesulfame, and combinations thereof.

The pH of the composition may be adjusted by the addition of suitable food acid, buffer, or both. In one embodiment, the nutraceutical composition may include a food acid. In one embodiment, the food acid may include malic acid, fumaric acid, lactic acid, tartaric acid, glucono-delta lactone, salts of gluconic acid, phosphoric acid, succinic acid, adipic acid, acetic acid, citric acid, or a combination thereof. In one embodiment, the nutraceutical composition may include from about 0% to about 0.4% ascorbic acid by weight.

In one embodiment, the pH of the composition may be from about 3 to about 5. In one embodiment, the pH of the composition may be from about 3 to about 4.5, or from about 3.7 to about 4. In one embodiment, the buffer may be sodium or potassium tartrate.

In one embodiment, the nutraceutical composition may include a flavoring agent. In one embodiment, the flavoring agent may include vanilla, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, or mixtures or derivatives thereof. In one embodiment, the flavoring agent may be an essential oil having estrogenic activity. In one embodiment, the flavoring agent may include Rosemary Oil, lavender oil, sage oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, or a combination thereof.

The nutraceutical composition may further comprise a bulking agent. The bulking agent may include maltitol syrup, polydextrose, sorbitol, soluble corn fiber, resistant starch, resistant maltodextrin, cellulose, hemicellulose, fructo-oligosaccharides, galacto-oligosaccharides, lactulose, xylo-isomalto-oligosaccharide, soybean oligosaccharide, oligo-glucose, stachyose, lactosucrose, or a combination thereof.

The nutraceutical composition may further comprise a preservative. The preservative may be any common food preservative. In one embodiment, the preservative may be potassium sorbate, sodium sorbate, sodium benzoate, potassium benzoate, citrate salts or a combination thereof.

In one embodiment, the nutraceutical composition may further include dietary fibers or prebiotics. In one embodiment, the dietary fiber is fructooligiosaccharides.

In another aspect, the application provides a nutraceutical product kit. In one embodiment the kit include a first product, comprising a neuroprotecting composition; a second product, comprising an anti-inflammatory composition; a third product, comprising a cardiovascular protecting composition, and a fourth product, comprising an antioxidant composition. In one embodiment, the nutraceutical product kit further comprises a fifth product, comprising a telomerase composition. In one embodiment, the nutraceutical product kit further comprises a sixth product, comprising a vitamin composition. In one embodiment, the nutraceutical product kit further comprises a seventh product, comprising a mineral composition. In one embodiment, the application provides a nutraceutical product kit, wherein each product is in a form selected from gummy, gel, tablet, capsule, soft-chew, powder, or a combination thereof. In one embodiment, the application provides a nutraceutical product kit, wherein each product is a gummy product.

In a further aspect, the application provides methods for preparing a nutraceutical composition. In one embodiment, the method includes the steps of: preparing a first premix by dissolving a mixture of ingredients comprising a sweetener, a gelling component, and water sufficiently into water; preparing a gelling solution by mixing a gelling composition and a nutraceutical mixture in water sufficiently to dissolve in the water; and blending the first premix and the gelling solution to form a combined mixture. In one embodiment, the method further comprise mixing the nutraceutical mixture in presence of at least one gelling component to provide a mixture, and pulverizing the mixture until an average particle diameter of reaches 50 um or less.

In a further aspect, the application provides methods for using the nutraceutical composition for managing health and/or delay aging effect. In one embodiment, the method includes the steps of providing the nutraceutical composition or kit disclosed thereof to a subject and providing the instruction to the subject to consuming such composition or kit. In one embodiment, the method includes the steps of providing the nutraceutical composition disclosed thereof and consuming such composition or kit for achieving health management or delay aging effect.

These and other objects and advantages of the application shall become apparent from the detailed description that follows.

### DETAILED DESCRIPTION

The application provides nutraceutical compositions facilitating health management, anti-aging, neuroprotecting and assisting in management metabolic syndromes. In one embodiment, the application provides a nutraceutical composition comprising a neuroprotecting composition, an anti-inflammatory composition, a cardiovascular protecting composition, an antioxidant composition, and a gelling component in a sufficient amount to provide a cohesive gelled product.

In one embodiment, the composition further includes a telomerase activating composition. In one embodiment, the composition further includes an amino acid composition. In one embodiment, the composition further includes a neuro-stimulant composition. In one embodiment, the composition further includes a vitamin composition. In one embodiment, the composition further includes a mineral composition.

Neuroprotecting Composition

Neuroprotecting agents refer to substances that are capable of preserving brain function and structure. Administration of a neuroprotecting agent may help minimize the effects of chronic conditions that could kill brain cells, decrease brain volume, or lead to long-term functional impairment.

Neuroprotecting agent generally works by altering the balance of particular chemicals (neurotransmitters) in the brain. Some acts by selective enhancement of cerebral blood flow, cerebral oxygen usage metabolic rate and cerebral glucose metabolic rate in chronic impaired human brain function i.e., multi-infarct (stroke) dementia, senile dementia of the Alzheimer type and pseudo dementia, ischaemic cerebral (poor brain blood flow) infarcts. One of the neuroprotecting mechanisms against dementia is decreased cholinergic activity in brain. Example cholinergic agents that may improve memory include muscarinic agonists (e.g. arecoline, pilocarpine etc.), nicotinic agonists (e.g. nicotine) and cholinesterase inhibitors (e.g. huperzine). Other neuroprotecting agents against dementia may include stimulants or nootropics (e.g. piracetam, amphetamine), putative cerebral vasodilators (e.g. ergot alkaloids, papavarine), and calcium channel blocker (e.g. nimodipine).

In one embodiment, the neuroprotective composition may include plant origined or herbal agents. Herbal products contain complicated mixtures of organic chemicals, which may include fatty acids, sterols, alkaloids, flavonoids, glycosides, saponins, tannins, terpenes and so forth. The plant's therapeutic value may come from the synergistic effects of the various components of the plants or herbs.

In one embodiment, the neuroprotecting composition comprises melatonin, L-carnitine, acetyl L-carnitine, alpha-lipoic acid, ginseng or its extract, or a combination thereof. In one embodiment, the neuroprotecting composition consists of melatonin, acetyl L-carnitine, alpha-lipoic acid, ginseng or its extract, or a combination thereof. In one embodiment, the ginseng comprises Asian ginseng (Panax ginseng), Siberian ginseng (Eleutherococcus senticosus), American ginseng (Panax quinquefolius), Indian ginseng (Withania somnifera), or a combination thereof.

Melatonin, chemically N-acetyl-5-methoxy tryptamine, is a substance found in animals, plants, fungi, and bacteria. In animals, it is a hormone that anticipates the daily onset of darkness. Melatonin is involved in the entrainment (synchronization) of the circadian rhythms of physiological functions including sleep timing, blood pressure regulation, seasonal reproduction, and many others. Many of melatonin's biological effects are produced through activation of melatonin receptors, while others are due to its role as a pervasive and powerful antioxidant, with a particular role in the protection of nuclear and mitochondrial DNA.

L-carnitine or Acetyl L-carnitine is an amino acid (a building block for proteins). L-Carnitine and the related compound Acetyl-L-Carnitine (ALCAR) are compounds able to alleviate the effects of aging and disease on mitochondria, while increasing the mitochondria's potential to burn fat. L-carnitine or Acetyl L-carnitine is often used as a brain booster, due to its ability to increase alertness and mitochondrial capacity while providing support for the neurons. L-carnitine or Acetyl L-carnitine has been shown to be effective at alleviating the side effects of aging, like neurological decline and chronic fatigue. The compound may also protect neurons and repair certain damage, such as that caused by diabetes and diabetic neuropathy.

Alpha-lipoic acid (LA), also known as α-lipoic acid and alpha lipoic acid (ALA) and thioctic acid, is an organosulfur compound derived from octanoic acid. Alpha-Lipoic Acid (ALA) is a mitochondrial fatty acid that is highly involved in energy metabolism. It has shown benefit against various forms of oxidation and inflammation. These effects carry on to benefits that protect one from heart diseases, liver diseases, diabetes, and neurological decline with age. It is a potent anti-oxidant compound. It works with mitochondria and the body's natural anti-oxidant defenses. It is also seen as an anti-aging compound since it can reverse some of the oxidant damage related effects of aging.

Ginseng consists of several species belonging to the plant family Araliaceae. The two major forms are Chinese, Korean or Asian ginseng that belong to the genus Panax, and Siberian or Russian ginseng that belongs to the genus Eleutherococcus. The biologically active constituents in Panax ginseng are a complex mixture of triterpene saponins known as ginsenosides. Siberian, or Russian, ginseng consists of the dried roots and rhizome of Eleutherococcus senticosis, and contains phenolics, polysaccharides, and eleutherosides. In China, Eleutherococcus senticosus is known as wujiaseng or Ciwujia with active ingredients as ciwujianosides. In some embodiments, the ginseng may be Asian ginseng (Panax ginseng), Siberian ginseng (Eleutherococcus senticosus), American ginseng (Panax quinquefolius), Indian ginseng (Withania somnifera), or its extracts or isolates thereof.

Anti-Inflammatory Composition

The anti-inflammatory composition includes agent that is capable of reduce or attenuate inflammation. In one embodiment, the anti-inflammatory composition comprises acetylsalicylic acid, willow or its extract, curcumin, ginger or its extract, or a combination thereof. In one embodiment, the anti-inflammatory composition consists essentially of acetylsalicylic acid, curcumin, and ginger root extract.

Acetylsalicylic acid (aspirin) is a nonsteroidal anti-inflammatory drug (NSAID). It is often used to treat pain, fever, and inflammation. The salicylates have similar effects (antipyretic, anti-inflammatory, analgesic) to the other NSAIDs and inhibit the same enzyme cyclooxygenase (COX), but aspirin does so in an irreversible manner and, unlike others, affects the COX-1 variant more than the COX-2 variant of the enzyme. Aspirin also has an antiplatelet effect by stopping the binding together of platelets.

Curcumin is the yellow pigment associated with the curry spice, Turmeric, and to a lesser extent Ginger. It is a small molecule that is the prototypical ‘curcuminoid’, and has effects similar to other polyphenols but unique in a way as it is a different class of polyphenol (relative to the other classes of ‘flavonoid’, ‘stilbene’, etc.) It exerts potent anti-inflammatory effects, and these anti-inflammatory effects seem to be quite protective against some form of cancer progression. However, curcumin has additional anti-cancer effects that are independent of its anti-inflammatory effects and thus is a heavily researched molecule for both cancer prevention and treatment.

Other areas of interest as it pertains to curcumin are alleviating cognitive decline associated with aging, being heart healthy by both electrical means and reducing lipid and plaque levels in arteries, and both reducing the risk of diabetes and being a good treatment for the side-effects associated with diabetes.

Ginger (Zingiber officinalis) is a powerful anti-inflammatory herb. It is used for its antispasmodic, anti-inflammatory, circulatory stimulant, antiemetic, carminative. Ginger is inotropic (promoting heart muscle tone) activities. Good for nausea and aids in digestion. As used herein and in the claims ginger is synonymous with ginger root.

Boswellia serrata (Salai/Salai guggul), is a moderate to large sized branching tree of family Burseraceae (Genus Boswellia). The oleo gum-resins contain 30-60% resin, 5-10% essential oils, and the rest is made up of polysaccharides. Gum-resin extracts of Boswellia serrata have been traditionally used in folk medicine for centuries to treat various chronic inflammatory diseases. The resinous part of Boswellia serrata possesses monoterpenes, diterpenes, triterpenes, tetracyclic triterpenic acids and four major pentacyclic triterpenic acids i.e. β-boswellic acid, acetyl-β-boswellic acid, 11-keto-β-boswellic acid and acetyl-11-keto-β-boswellic acid, responsible for inhibition of pro-inflammatory enzymes. Out of these four boswellic acids, acetyl-11-keto-β-boswellic acid is the most potent inhibitor of 5-lipoxygenase, an enzyme responsible for inflammation.

Cardiovascular Protecting Composition

In one embodiment, the cardiovascular protecting composition comprises omega-3 fatty acids. Example omega-3 fatty acid includes alpha-linolenic acid (ALA), eicosapentaenoic acid (EPA) and docosahexaenoic acid (DHA). Once eaten, the human body converts ALA to EPA and DHA, which are more readily used by the body. Omega-3 is an important component of almost all cell membranes; therefore, sufficient amounts of these fatty acids are necessary and need to be balanced to maintain good health.

In one embodiment, the cardiovascular protecting composition comprises cod liver oil, flax seed oil, or a combination thereof. In one embodiment, cardiovascular protecting composition comprises fish oil, algae oil, cod liver oil, flax seed oil, or a combination thereof.

Cod liver oil is oil derived from liver of cod fish (Gadiadae). It has high levels of the omega-3 fatty acids, eicosapentaenoic acid (EPA) and docosahexaenoic acid (DHA). Cod liver oil also contains vitamin A and vitamin D. According to the United States Department of Agriculture, a tablespoon (4 drams or 15 ml) of cod liver oil contains 4080 μg of retinol (vitamin A) and 34 μg of vitamin D. Rich in omega-3 fatty acids, cod liver oil may be beneficial in secondary prophylaxis after a heart attack.

Flax seed oil (also called linseed oil) comes from the seeds of the flax plant. According to the Thomas Jefferson Agricultural Institute, a flax seed is about 40% oil by weight. Of this oil, 55% of it is omega-3 fatty acid.

Antioxidants Composition

In one embodiment, the antioxidant composition comprises bioflavonoids, pomegranate extract, Ginkgo biloba, green tea extract, garlic extract, grapeseed extract, vitamin E, vitamin C, resveratrol, coenzyme Q10, quercetin, rutin, lycopene, L-glutathione, N-acetyl cysteine, or a combination thereof. In one embodiment, the antioxidant composition further comprises phenolics, anthocyanins, flavonoids, anthracenes, carotenoids, zeaxanthin, blackberry extract, elderberry extract, cranberry extract, blueberry extract, saffron, Sangre de grado (dragon's blood) or derivatives thereof.

In some embodiments, the antioxidant composition may include but are not limited to ascorbic acid, citric acid, rosemary oil, vitamin A, vitamin E, vitamin E phosphate, tocopherols, di-alpha-tocopheryl phosphate, tocotrienols, coenzyme Q10, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and combinations thereof. In some embodiments, the antioxidant composition may include extracts and active phytochemicals such as ferulic acid (from apples), ginseng, Gingko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine, glucosamine, chondroitin, msm, melatonin, serotonin, and mixtures thereof.

In some embodiments, the antioxidant composition consists essentially of vitamin E, beta-carotene, and vitamin C. In some embodiments, per daily dosage, the composition includes at least 80 mg, 100 mg, 150 mg, 200 mg, 250 mg, 400 mg, or 500 mg of vitamin E. In some embodiments, per daily dosage, the composition includes at least 15 mg, 20 mg, 30 mg, or 50 mg of beta-carotene. In some embodiments, per daily dosage, the composition includes at least 500 mg, 600 mg, 800 mg, 1 g or 1.5 g of vitamin C.

Bioflavonoids are a class of nutrients found in plants. Chemically, they have the general structure of a 15-carbon skeleton, which consists of two phenyl rings (A and B) and heterocyclic ring (C). This carbon structure can be abbreviated C6-C3-C6. According to the IUPAC nomenclature, they can be classified into: flavonoids or bioflavonoids; isoflavonoids (derived from 3-phenylchromen-4-one (3-phenyl-1,4-benzopyrone) structure) and neoflavonoids (derived from 4-phenylcoumarine (4-phenyl-1,2-benzopyrone) structure).

Pomegranate extract contains a variety of chemicals that might have antioxidant effects including punicalagins. Punicalagins are really big molecules found in pomegranate juice that are somehow absorbed; they are potent anti-oxidants, and alongside Punicic Acid confer many of the benefits associated with pomegranates. Punicalagins are very potent anti-oxidants, and they can be metabolized into other compounds (ellagic acid, urolithins) that themselves have anti-oxidant capabilities (although lesser). This large anti-oxidant value of punicalagins is about thrice that of red wine and green tea. The metabolites of punicalagins, ellagic acid and urolithins, confer a lot of health benefits, but their anti-oxidant capabilities are on par with green tea and red wine if not a bit less potent.

Ginkgo biloba (Gb) has demonstrated antioxidant and vasoactive properties as well as clinical benefits. Extract from Gb leaves has been used in traditional Chinese medicine for centuries to treat circulatory disorders, asthma, tinnitus, vertigo, and cognitive problems. Gb extract contains mainly terpenoids, flavonol glycosides, and proanthocyanidins. The most prevalent of these three groups are the flavonol glycosides (quercetin, catechin). The terpenoids include ginkgolides and bilobalides, which represent unique components of Gb. Terpenoids, flavonoids and proanthocyanidins are thought to be responsible for the pharmacological properties of Gb. Extract from Gb leaves inhibits platelet-activating factor and enhances NO production in vessels, with subsequent effect on peripheral and cerebral blood flow. Gb extract is thought to module different neurotransmitter systems: it is a strong inhibitor of monoamine oxidase A and synaptosomal uptake of DA, 5-HT, and norepinephrine. Additionally, Gb displays a free radical scavenger activity and has neuroprotecting and antiapoptotic properties, such as inhibition of amyloid-β neurotoxicity and protection against hypoxic challenges and increased oxidative stress.

Green tea is made from the leaves of the Camellia sinensis L plant, which is rich in polyphenol catechins and caffeine. Green tea provides health benefits for a wide variety of ailments, including the prevention of cancer and cardiovascular diseases, the anti-inflammatory, antiarthritic, antibacterial, antiangiogenic, antioxidative, antiviral, neuroprotecting, and cholesterol-lowering effects. The health-promoting effects of green tea are mainly attributed to its polyphenol content, particularly flavanols and flavonols, which represent 30% of fresh leaf dry weight. Recently, many of the aforementioned beneficial effects of green tea were attributed to its most abundant catechin, (−)-epigallocatechin-3-gallate (EGCG). Green tea extracts are more stable than pure epigallocatechin gallate, one of the major constituents of green tea, because of the presence of other antioxidant constituents in the extract.

Garlic ranks highly among health protecting foods, largely due to its antioxidant sulfur compounds. Fresh garlic, however, is not for everyone; it can cause indigestion and its odor is a possible social deterrent. The health benefits of garlic, however, do not depend on freshness or pungency. Scientific studies show that aged garlic extract (AGE), which is odorless and richer in antioxidants than fresh or other forms of garlic preparations, is more effective in boosting immunity and protecting against cardiovascular disease, cancer, aging, and drug toxicity.

Resveratrol (3,5,4′-trihydroxystilbene) is a plant-derived polyphenol. It is an antioxidant and has been shown in vitro as an effective scavenge for free radicals. Resveratrol was found to induce antioxidant enzymes, including superoxide dismutase (SOD), thioredoxin, glutathione peroxidase-1, heme oxygenase-1, and catalase, and/or inhibit reactive oxygen species (ROS) production by nicotinamide adenine dinucleotide phosphate (NADPH) oxidases (NOX). It has been shown to possess numerous biological functions, which could possibly be applied to the prevention and/or treatment of cancer, cardiovascular disease, and neurodegenerative diseases.

CoEnzyme Q10, also known as ubiquinone, is an antioxidant that is made in the human body. CoQ10 is needed for basic cell function. This fat-soluble substance, which resembles a vitamin, is present in most eukaryotic cells, primarily in the mitochondria. CoQ10 levels decrease with age and may be low in people with cancer, certain genetic disorders, diabetes, heart conditions, HIV/AIDS, muscular dystrophies, and Parkinson's disease.

Quercetin is a flavonol found in many fruits, vegetables, leaves and grains. It has antioxidant and anti-inflammatory effects.

Rutin, also called rutoside, quercetin-3-O-rutinoside and sophorin, is the glycoside between the flavonol quercetin and the disaccharide rutinose (α-L-rhamnopyranosyl-(1→6))-β-D-glucopyranose). Rutin is a citrus flavonoid glycoside. It is an antioxidant with anti-inflammatory and antinociceptive activities.

Lycopene is a bright red carotene and carotenoid pigment and phytochemical found in tomatoes and other red fruits and vegetables, such as red carrots, watermelons, gac, and papayas. An antioxidant, lycopene does exhibit a physical quenching rate constant with singlet oxygen almost twice as high as that of beta-carotene.

Glutathione (GSH) is an important antioxidant in plants, animals, fungi, and some bacteria and archaea. GSH is capable of preventing damage to important cellular components caused by reactive oxygen species such as free radicals, peroxides, lipid peroxides and heavy metals. It is a tripeptide with a gamma peptide linkage between the carboxyl group of the glutamate side chain and the amine group of cysteine, and the carboxyl group of cysteine is attached by normal peptide linkage to a glycine.

Thiol groups are reducing agents, existing at a concentration around 5 mM in animal cells. Glutathione reduces disulfide bonds formed within cytoplasmic proteins to cysteines by serving as an electron donor. In the process, glutathione is converted to its oxidized form, glutathione disulfide (GSSG), also called L-(−)-glutathione. Once oxidized, glutathione can be reduced back by glutathione reductase, using NADPH as an electron donor. The ratio of reduced glutathione to oxidized glutathione within cells is often used as a measure of cellular oxidative stress.

N-Acetylcysteine (NAC) is a prodrug for L-cysteine, which is used for the intention of allowing more glutathione to be produced when it would normally be depleted. Through glutathione buffering, NAC provides anti-oxidative effects and other benefits.

Telomerase Activating Composition

In one embodiment, the telomerase activating composition is capable of stimulating telomerase activity. Telomerase is a species-specific ribonucleoprotein that catalyzes the addition of oligonucleotide repeats (telomeres) onto the ends of chromosomal DNAS, to compensate for losses that occur with each round of DNA replication. Somatic cells, in contrast to tumor cells and certain stem cells, have little or no telomerase activity and stop dividing when the telomeric ends of at least some chromosomes have been shortened to a critical length, leading to programmed cellular senescence (cell death). Accordingly, an increase in telomeras activity is expected to inhibit cell death, and to promote cell proliferation.

In one embodiment, the telomerase activating composition comprises Astragalus, extracts or isolates thereof. Astragalus (Astragalus membranaceus and Astragalus mongholicus, huang-qi) has been used in Chinese traditional medicine for thousands of years for supporting and enhancing the immune system. The herb contains polysaccharides, saponins, flavonoids, anthraquinones, alkaloids, beta-sitosterol, amino acids and trace elements. Researches have shown that two isomers of HDTIC compounds from Astragali Radix, 4-hydroxy-5-hydroxymethyl-[1,3]dioxolan-2,6′-spirance-5′,6′,7′,8′-tetrahydro-indolizine-3′-carbaldehyde (HDTIC)-1 and HDTIC-2, slow down telomere shortening via attenuating oxidative stress and increasing DNA repair ability. In addition, cycloastragenol (CAG), an aglycone of astragaloside IV from Astragalus membranaceus extracts, as well as astragaloside IV have been shown to stimulate telomerase activity and cell proliferation in human neonatal keratinocytes.

A. membranceus, A. mongholicus and A. complanatus have been mainly used in folk medicine for their anti-inflammatory, immune-stimulant, anti-oxidative, anti-cancer, antidiabetic, anti-aging, cardioprotective, hepatoprotective, neuron protective, antiviral properties. The active constituents for the above-mentioned effects were polysaccharides, saponins and flavonoids.

The herbal component may include the powder of the whole herb or any part thereof including stems, leaves, roots, fruits, or flowers, or extracts or isolates of the whole herb or any parts thereof. The extract may be a hydrophilic extract or lipophilic extract. The hydrophilic extract may be an extract through extraction with hydrophilic solvents including without limitation water, alcohol, acetone, or a combination thereof. The hydrophobic extract may be an extract through extraction with hydrophobic solvents including without limitation ether, ethyl acetate, methylene chloride, or a combination thereof.

Vitamin Composition

In some embodiments, the vitamin composition may include fat-soluble vitamins. Example fat-soluble vitamins include vitamin A, vitamin D, vitamin E, and vitamin K and combinations thereof. In some embodiments, the vitamin composition may include water-soluble vitamins. Example water-soluble vitamins include vitamin C (ascorbic acid), the B vitamins, and choline.

In some embodiment, the composition comprises Vitamin B1, Vitamin B3, Vitamin B6, Vitamin B12, Folic acid, Vitamin C, Vitamin D, Vitamin E, beta-carotene, lutein or a combination thereof.

B vitamins are a class of water-soluble vitamins that play important roles in cell metabolism. In some embodiments, the composition includes a vitamin composition. In some embodiments, the Vitamin composition includes folate, B6, and B12. In some embodiments, the vitamin B composition consists of thiamine (B1), riboflavin (B2), niacin (B3), panotothenic acid (B5), pyridoxine (B6), biotin (B7), folic acid (B9), cobalamins (B12), derivatives or combinations thereof. In some embodiments, per daily dosing, the nutraceutical composition comprises at least 300 mcg, 400 mcg, 450 mcg, or 500 mcg of folate, at least 3 mg, 4.5 mg, 5.5 mg, 6.5 mg of B1, at least 3 mg, 5 mg, 6 mg, or 7 mg of B2, at least 12 mg, 15 mg, 16 mg, 18 mg, or 20 mg of niacinamide, at least 4 mg, 6 mg, 7 mg, 8 mg, 10 mg of B6, at least 15 mcg, 20 mcg, 30 mcg, 50 mcg, 1 mg, 2 mg, 4 mg, 5 mg, or 6 mg of B12, at least 30 mcg, 40 mcg, 50 mcg, 80 mcg, or 1 mg of biotin, or at least 7 mg, 10 mg, 12 mg, 15 mg, or 20 mg of pantothenic acid.

Vitamin D refers to a group of fat-soluble secosteroids responsible for increasing intestinal absorption of calcium, iron, magnesium, phosphate, and zinc. In humans, the most important compounds in this group are vitamin D3 (also known as cholecalciferol) and vitamin D2 (ergocalciferol). Very few foods contain vitamin D; synthesis of vitamin D (specifically cholecalciferol) in the skin is the major natural source of the vitamin. Dermal synthesis of vitamin D from cholesterol is dependent on sun exposure (specifically UVB radiation). Adequate vitamin D intake reduces risk for conditions such as stress fracture, total body inflammation, infectious illness, and impaired muscle function. In some embodiments, per daily dosage, the composition includes at least 400 IU, 600 IU, 800 IU, 1000 IU, 1200 IU, 1500 IU, or 2000 IU of Vitamin D.

Vitamin A is important for growth and development, for the maintenance of the immune system and good vision. Vitamin A is needed by the retina of the eye in the form of retinal, which combines with protein opsin to form rhodopsin, the light-absorbing molecule necessary for both low-light (scotopic vision) and color vision. Vitamin A also functions in a very different role as retinoic acid (an irreversibly oxidized form of retinol), which is an important hormone-like growth factor for epithelial and other cells. In some embodiments, per daily dosage, the nutraceutical composition includes at least 500 IU, 800 IU, 1000 IU, 1500 IU, 2000 IU, 2500 IU or 3000 IU of Vitamin A.

Vitamin C is a cofactor in enzymatic reactions, including several collagen synthesis reactions that, when dysfunctional, cause the most severe symptoms of scurvy. In animals, these reactions are especially important in wound-healing and in preventing bleeding from capillaries. Ascorbate also acts as an antioxidant, protecting against oxidative stress. In some embodiments, per daily dosage, the nutraceutical composition includes at least 10 mg, 20 mg, 40 mg, 60 mg, 100 mg, 500 mg, 800 mg, 1000 mg, or 1500 mg of Vitamin C.

Vitamin E refers to a group of compounds that include both tocopherols and tocotrienols. As a fat-soluble antioxidant, it stops the production of reactive oxygen species formed when fat undergoes oxidation. In some embodiments, per daily dosage, the nutraceutical composition includes at least 10 IU, 20 IU, 30 IU, 40 IU, 50 IU or 100 IU of Vitamin E.

Vitamin K is a group of structurally similar, fat-soluble vitamins the human body requires for complete synthesis of certain proteins that are prerequisites for blood coagulation that the body needs for controlling binding of calcium in bones and other tissues. The vitamin K-related modification of the proteins allows them to bind calcium ions, which they cannot do otherwise. Without vitamin K, blood coagulation is seriously impaired, and uncontrolled bleeding occurs. Low levels of vitamin K also weaken bones and promote calcification of arteries and other soft tissues. Chemically, the vitamin K family comprises 2-methyl-1,4-naphthoquinone (3-) derivatives. Vitamin K includes two natural vitamers: vitamin K1 and vitamin K2. Vitamin K2, in turn, consists of a number of related chemical subtypes, with differing lengths of carbon side chains made of isoprenoid groups of atoms. In some embodiments, per daily dosage, the nutraceutical composition includes at least 10 mcg, 15 mcg, 20 mcg, 30 mcg, 40 mcg, 50 mcg of Vitamin K.

Beta-carotene is a member of the carotenes, which are terpenoids (isoprenoids), synthesized biochemically from eight isoprene units and thus having 40 carbons. β-carotene is a precursor (inactive form) to vitamin A via the action of beta-carotene 15,15′-monooxygenase. Beta-carotene is used to decrease asthma symptoms caused by exercise; to prevent certain cancers, heart disease, cataracts, and age related macular degeneration (AMD); and to treat AIDS, alcoholism, Alzheimer's disease, depression, epilepsy, headache, heartburn, high blood pressure, infertility, Parkinson's disease, rheumatoid arthritis, schizophrenia, and skin disorders including psoriasis and vitiligo.

Lutein is called a carotenoid vitamin. It is related to beta-carotene and vitamin A. Lutein is suggested to be employed by animals as an antioxidant and for blue light absorption.

Minerals Composition

In one embodiment, the mineral composition may include potassium, chromium pocolinate, magnesium and selenium. In one embodiment, the mineral composition may include ions of sodium, magnesium, chromium, iodine, iron, manganese, calcium, copper, fluoride, potassium, phosphorous, molybdenum, selenium, zinc, and combinations thereof. The minerals may be in the forms of salts or chelates.

Chromium pocolinate: Chromium(III) picolinate (CrPic3) is a bright-red coordination compound derived from chromium(III) and picolinic acid. Small quantities of chromium are needed for glucose utilization by insulin in normal health. Chromium has been identified to regulate insulin by increasing the sensitivity of the insulin receptor.

Selenium is a trace element. It is a constituent of more than two dozen selenoproteins that play critical roles in reproduction, thyroid hormone metabolism, DNA synthesis, and protection from oxidative damage and infection and therefore is nutritionally essential for humans.

Magnesium is involved in adenosine triphosphate (ATP) production from fatty acid oxidation, post-contractile muscular relaxation, and bone remineralization. It is also involved in phosphatidylglycerol (DPG) production, which is important to red blood cell formation. ATP, present in all cells but particularly in muscle cells, stores energy. Athletes lose magnesium through sweat and urine. In some embodiments, per daily dosage, the composition includes at least 400 mg, 500 mg, or 800 mg of magnesium.

Essential to antioxidant glutathione peroxidase (SeGPx) production, selenium is a free radical-scavenging tripeptide made up of glutamine, cysteine and glycine. It is concentrated in the lining of the GI tract and lungs, in the liver, and in skeletal muscle. Reducing muscular SeGPx increased cellular damage from prolonged exercise, supporting the theory that free radical-induced muscle damage causes muscle fatigue. In addition, selenium benefits athletes' immune function and helps repair cellular damage. In some embodiments, per daily dosage, the composition includes at least 150 mcg, 200 mcg, or 250 mcg of selenium.

Zinc aids in post-exertion tissue repair and in the conversion of food to fuel. Example zinc compounds include Zinc picolinate and monomethionate. In some embodiments, per daily dosage, the composition includes at least 25 mg, 30 mg, 50 mg, 60 mg, or 70 mg of Zinc.

In some embodiments, per daily dosage, the composition includes from at least 800 mg, 1000 mg, 1200 mg, or 1500 mg of calcium. In some embodiment, per daily dosage, the composition includes at least 10 mg, 15 mg, 20 mg, or 30 mg of iron.

In another embodiment, the composition may further include a neuro-stimulant composition. In one embodiment, the composition may further include an energy composition. In some embodiments, the nutraceutical composition may further include additives, including without limitation, sweeteners, food acids, flavoring agents, coloring agents, sensate agents, freshening agents, probiotics, prebiotics, bulking agents, hemectants, plasticizers, preservatives, stabilizing agents, emulsifiers, and thickening agents.

Neuro-Stimulant Composition

The nutraceutical composition may include a neuro-stimulant composition. The stimulant composition may include without limitation caffeine, coffee, guarana, kola nut, mate (Ilex paraguariensis), licorice, ephedra (Ephedra sinica or ma-huang), synepherine (Citrus aurantium), green tea (Camellia sinensis), or extracts or isolates thereof.

Fibers and Prebiotics

The composition may further include fibers or prebiotics. In one embodiment, the fibers may present in an amount of from about 0.001% to 80%, alternatively 0.001% to 5%, alternatively 5% to 10%, alternatively 10% to 15%, alternatively 15% to 40%, alternatively 40% to 60%, alternatively 60% to 80%, by weight of said composition. Any suitable fiber can be used. In one embodiment, a naturally derived fiber is used, including one or more selected from naturally derived inulin, inulin extract, synthetic inulin, hydrolysis products of inulin commonly known as fructooligosaccharides, galacto-oligosaccharides, xylooligosaccharides, oligo derivatives of starch, husks, brans, psyllium, polysaccharides, starches, polycarbophil, lignin, arabinogalactans, chitosans, oat fiber, soluble corn fiber, non-digestible corn dextrin, non-digestible wheat dextrin, locust bean gum and derivatives of locust bean gum, hydroxypropylmethyl cellulose (HPMC), pectin, and mixtures thereof.

In some embodiments, fibers may include inulin, wheat dextrin, or fructooligiosaccharides. Inulin, wheat dextrin, and fructooligiosaccharides may also act as a thickening agent and improve the texture of the composition. Various load rates of dietary fiber can be incorporated in the composition to create improved texture and at certain load rates can provide dietary benefits including promoting a healthy digestion system, controlling blood sugar levels, and providing probiotic benefits. The addition of the dietary fiber along with the remaining components allow for the addition of water that helps displace sugar within the flavored chewy or gummy confection.

Inulin is indigestible by human enzymes ptyalin and amylase, which are designed to digest starch. As a result, inulin passes through much of the digestive system intact. Inulin is a highly effective prebiotic, stimulating the growth of beneficial probiotic bacteria in the gut. Inulin is used in low fat products because of its ability to provide a creamy smooth texture to products. Inulin is a dietary fiber and is believed to activate beneficial good bacteria in the digestive tract. The activation of these bacteria is thought to reduce the risk of bowel cancer. Inulin has a mildly sweet taste, but does not affect blood sugar levels and is recommended for diabetics. Inulin has been clinically proven to increase calcium absorption. The inherent calcium in dairy foods is now an even better source of this bone-building mineral when inulin is added because inulin improves the body's uptake. People have used plants containing inulin to help relieve diabetes mellitus, a condition characterized by hyperglycemia and/or hyperinsulinemia. The embodiments can provide flavored chewy or gummy candy with inulin as the fiber component and calcium supplementation. This flavored chewy or gummy candy would have an improved absorption of calcium because of the inulin within its composition. Calcium is one of the top deficient minerals in the diet of most individuals, and products produced in accordance with the guidelines provided herein can provide great tasting flavored chewy or gummy confections that can help consumers get more calcium within their diets by eating our products.

In some embodiments, the composition may further include probiotics, Example probiotics include, but not limited to, lactic acid producing microorganisms such as Bacillus coagulans, Bacillus subtilis, Bacillus laterosporus, Bacillus laevolacticus, Sporolactobacillus inulinus, Lactobacillus acidophilus, Lactobacillus curvatus, Lactobacillus plantarum, Lactobacillus jenseni, Lactobacillus casei, Lactobacillus fermentum, Lactococcus lactis, Pedioccocus acidilacti, Pedioccocus pentosaceus, Pedioccocus urinae, Leuconostoc mesenteroides, Bacillus coagulans, Bacillus subtilis, Bacillus laterosporus, Bacillus laevolacticus, Sporolactobacillus inulinus and mixtures thereof. Breath fresheners are also known by the following trade names: Retsyn,™ Actizol,™ and Nutrazin.™ Examples of malodor-controlling compositions are also included in U.S. Pat. No. 5,300,305 to Stapler et al. and in U.S. Patent Application Publication Nos. 2003/0215417 and 2004/0081713 which are incorporated in their entirety herein by reference for all purposes.

Sweetener

In general, an effective amount of sweetener may be utilized to provide the level of sweetness desired, and this amount may vary with the sweetener selected. In some embodiments the amount of sweetener may be present in amounts from about 0.001% to about 3%, by weight of the composition, depending upon the sweetener or combination of sweeteners used. The exact range of amounts for each type of sweetener may be selected by those skilled in the art.

Sweeteners may include one or more monosaccharides or disaccharides. Examples include sugar, sucrose, invert sugar, dextrose, lactose, honey, malt syrup, malt syrup solids, maltose, fructose, granular fructose, maple syrup, rice syrup, rice syrup solids, sorghum syrup, refiners syrup, corn syrup, corn syrup solids, high fructose corn syrup, molasses, or combinations thereof.

In one embodiment, the sweetener include common sugars such as sucrose and glucose, polyols such as maltitol, erythritol, and isomalt, syrup sweeteners such as glucose syrup, corn syrup, high fructose corn syrup, and juice concentrates.

The amount of sweetener can range from 20 to 60%, 40 to 80% by weight or from 50 to 70% by weight based on the total weight of the composition. In one embodiment, the composition contains about 30 to about 80 wt %, or about 45 wt %, glucose syrup. The glucose syrup may be combined with about 0 to about 25 wt %, or about 19 wt %, water. In one embodiment, the composition may contain about 0 to about 50 wt %, or about 30 wt % common sugar. In one embodiment, the weight ratio of sucrose to corn syrup solids will be within the range of from about 1:2 to about 2:1, or from about 2:3 to 3:2. The sucrose can be employed in any suitable physical form to achieve the batching requirements. Typically, the sucrose will be granular and the corn syrup will preferably be added as a liquid having a DE of greater than 42, e.g., 63 DE. The higher DE values will decrease the tendency for granulation and aid in maintaining softness.

In one embodiment, artificial sweeteners can be used such as acesulfame K, aspartame, sucralose, d-tagatose, neotame, monatin, and acesulfame potassium (Ace-K), or combinations thereof.

The sweeteners involved may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:

(a) water-soluble sweetening agents such as dihydrochalcones, monellin, steviosides, lo han quo, lo han quo derivatives, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame), N—[N-(3,3-dimethylbutyl)-L-.alpha.-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro 1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideo-xy-beta-D-fructofuranoside, or 4,6,1′,6′-tetrachloro-4,6, 1′,6′-tetradeoxygalacto-sucrose; and 4,6,1′,6′-tetradeoxy-sucrose, and mixtures thereof;

(e) protein based sweeteners such as Thaumaoccous danielli (Thaumatin I and II) and talin; and

(f) the sweetener monatin (2-hydroxy-2-(indol-3-ylmethyl)-4-aminoglutaric acid) and its derivatives.

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In some embodiments, the sweetener may be a polyol. Polyols can include, but are not limited to glycerol, sorbitol, maltitol, maltitol syrup, mannitol, isomalt, erythritol, xylitol, hydrogenated starch hydrolysates, polyglycitol syrups, polyglycitol powders, lactitol, and combinations thereof.

In general, an effective amount of intense sweetener may be utilized to provide the level of sweetness desired, and this amount may vary with the sweetener selected. The intense sweetener may be present in amounts from about 0.001% to about 3%, by weight of the composition, depending upon the sweetener or combination of sweeteners used. The exact range of amounts for each type of sweetener may be selected by those skilled in the art.

Food Acids

The pH of the composition is about 3 to about 5, about 3.7 to about 4. The pH may be adjusted by a food acid, buffer, or both.

Suitable food acids include but are not limited to acetic acid, adipic acid, ascorbic acid, butyric acid, citric acid, formic acid, fumaric acid, glyconic acid, lactic acid, phosphoric acid, malic acid, oxalic acid, succinic acid, tartaric acid, or combinations thereof.

Suitable buffers include but are not limited to sodium citrate and potassium citrate. For example, an acid/buffer system is 1.33% of a 54% citric acid solution, buffered with sodium citrate.

The amount of acid will be in the typical range of from about 0.5 to about 2% by weight, e.g., about 1.25%. Higher acid (lower pH) results in a lack of structure while lower acid levels do not provide enough “acid bite” in the flavor profile.

Flavoring Agents

In some embodiments, the composition may further include a flavoring agent. Flavoring agents may include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof.

In some embodiments, the flavoring agents may include mint(s), menthol, menthone, isomenthone, camphor and eucalyptol, eucalyptol, camphor, borneol, fenchone, menthone and isomenthone, isopulegol, monomenthyl succinate, and menthyl lactate, menthone, isomenthone, borneol, fenchone, eucalyptus, ducalyptol, ethyl benzoate, neomenthol, d-fenchone, furfurylidene butyrate, bucchu fractions, sage oil, corn mint oil, rosemary, monomenthyl succinate, amyl salicylate, eugenol, phellendrene, propyl furoate, ethyl-3-hydroxy butyrate, hexyl valerate, anisyl propionate, anysyl butyrate, dihydrocarveol, or clary sag,

Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors whose release profiles can be managed include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below. In some embodiments, flavorants may chose from geraniol, linalool, nerol, nerolidal, citronellol, heliotropine, methyl cyclopentelone, ethyl vanillin, maltol, ethyl maltol, furaneol, alliaceous compounds, rose type compounds such as phenethanol, phenylacetic acid, nerol, linalyl esters, jasmine, sandlewood, patchouli, and/or cedarwood.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, e.g., melonal (melon), 2,6-dimethyloctanal (green fruit), and 2-dodecenal (citrus, mandarin), cherry, grape, blueberry, blackberry, strawberry shortcake, and mixtures thereof.

In some embodiments, flavoring agents are used at levels that provide a perceptible sensory experience i.e. at or above their threshold levels. In other embodiments, flavoring agents are used at levels below their threshold levels such that they do not provide an independent perceptible sensory experience. At subthreshold levels, the flavoring agents may provide an ancillary benefit such as flavor enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

Illustrations of the encapsulation of flavors as well as other additional components can be found in the examples provided herein. Typically, encapsulation of a component will result in a delay in the release of the predominant amount of the component during consumption of a composition that includes the encapsulated component (e.g., as part of a delivery system added as an ingredient to the chewing composition). In some embodiments, the release profile of the ingredient (e.g., the flavor, sweetener, etc.) can be managed by managing various characteristics of the ingredient, delivery system containing the ingredient, and/or the composition containing the delivery system and/or how the delivery system is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the composition, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc.

Sensate Agents

In some embodiment, the composition further includes a sensate agent. Sensate agents can include cooling agents, warming agents, tingling agents, effervescent agents, and combinations thereof. A variety of cooling agents may be employed. For example, among the useful cooling agents are included xylitol, erythritol, dextrose, sorbitol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N,2,3-trimethyl-2-isopropyl butanamide (WS-23), N-ethyl-p-menthane-3-carboxamide (WS-3), isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl)-butanamide, n-ethyl-t-2-c-6 nonadienamide, N,N-dimethyl menthyl succinamide, substituted p-menthanes, substituted p-menthane-carboxamides, 2-isopropanyl-5-methylcyclohexanol (from Hisamitsu Pharmaceuticals, hereinafter “isopregol”); menthone glycerol ketals (FEMA 3807, tradename FRESCOLAT® type MGA); 3-1-menthoxypropane-1,2-diol (from Takasago, FEMA 3784); and menthyl lactate; (from Haarman & Reimer, FEMA 3748, tradename FRESCOLAT® type ML), WS-30, WS-14, Eucalyptus extract (p-Mentha-3,8-Diol), Menthol (its natural or synthetic derivatives), Menthol PG carbonate, Menthol EG carbonate, Menthol glyceryl ether, N-tertbutyl-p-menthane-3-carboxamide, P-menthane-3-carboxylic acid glycerol ester, Methyl-2-isopryl-bicyclo (2.2.1), Heptane-2-carboxamide; and Menthol methyl ether, and menthyl pyrrolidone carboxylate among others. These and other suitable cooling agents are further described in the following U.S. patents, all of which are incorporated in their entirety by reference hereto: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation can be provided. One such tingling sensation is provided by adding jambu, oleoresin, or spilanthol to some examples. In some embodiments, alkylamides extracted from materials such as jambu or sanshool can be included. Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of “tingling” type sensates can be found in U.S. Pat. No. 6,780,443, the entire contents of which are incorporated herein by reference for all purposes.

Sensate agents may also be referred to as “trigeminal stimulants” such as those disclosed in U.S. Patent Application No. 205/0202118, which is incorporated herein by reference. Trigeminal stimulants are defined as an orally consumed product or agent that stimulates the trigeminal nerve. Examples of cooling agents which are trigeminal stimulants include menthol, WS-3, N-sub stituted p-menthane carboxamide, acyclic carboxamides including WS-23, methyl succinate, menthone glycerol ketals, bulk sweeteners such as xylitol, erythritol, dextrose, and sorbitol, and combinations thereof. Trigeminal stimulants can also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin and menthol glycerin ether, unsaturated amides, and combinations thereof.

In some embodiments, sensate agents are used at levels that provide a perceptible sensory experience i.e. at or above their threshold levels. In other embodiments, sensate components are used at levels below their threshold levels such that they do not provide an independent perceptible sensory experience. At subthreshold levels, the sensates may provide an ancillary benefit such as flavor or sweetness enhancement or potentiation.

Freshening Agents

In some embodiments, the composition further includes a freshening agent. Freshening agents may include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments freshening agent can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof.

Coloring Agents

Coloring agents may be used in amounts effective to produce the desired color. The coloring agents may include pigments which may be incorporated in amounts up to about 6%, by weight of the composition. For example, titanium dioxide may be incorporated in amounts up to about 2%, and preferably less than about 1%, by weight of the composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-p-sulfoniumbenzylamino) diphenylmethylene]-[1-(N-ethyl-N-p-sulfoniumbenzyl)-delta-2,5-cyclohexadi-eneimine]. A full recitation of all F.D.& C. colorants and their corresponding chemical structures may be found in the Kirk-Othmer Encyclopedia of Chemical Technology, 3rd Edition, in volume 5 at pages 857-884, which text is incorporated herein by reference.

In some embodiments, one or more colors can be included. As classified by the United States Food, Drug, and Cosmetic Act (21 C.F.R. 73), colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), .beta.-apo-8′-carotenal (E160e), .beta.-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colors can include FD&C aluminum lakes. These include of the aluminum salts of FD&C dyes extended on an insoluble substrate of alumina hydrate. Additionally, in some embodiments, certified colors can be included as calcium salts.

In some embodiments, natural fruits or plant juice or extracts may be used as the coloring agents. Example include without limitation carrot juice, raspberry juice, blackberry juice, blueberry juice, and beet juice.

Plasticizer

In some embodiments, the composition may further include plasticizer to modify the texture of the formulation. A texture modifying agent may include a particulate material. Suitable particulate materials can include, but are not limited to, sucrose, polyols such as sorbitol, xylitol, mannitol, galactitol, lactitol, maltitol, erythritol, isomalt, hydrogenated starch hydrolysates and mixtures thereof, starches, proteins, and combinations thereof. In some embodiments, the particulate material serving as a texture modifying component is selected based on its ability or lack of ability to crystallize the saccharides in the saccharide portion. For example, when isomalt is included in the saccharide portion, sorbitol powder can be added to the composition because it will not cause the isomalt to crystallize. Alternatively, when erythritol is included in the saccharide portion, erythritol powder can be added to the composition because it will cause the erythritol to crystallize. Such particulates can be included in amounts from 5% to 35% w/w of the composition.

Fats and Oils

In some embodiments, a texture modifying component can include fats, oils, or other hydrophobic materials. Suitable fats can include, but are not limited to, partially hydrogenated vegetable or animal fats, such as coconut oil, corn oil, palm kernel oil, peanut oil, soy bean oil, sesame oil, cottonseed oil, cocoa butter, milk fat, beef tallow, and lard, among others. Suitable hydrophobic materials include chocolate, chocolate crumb, carob coatings, and compound coatings. Such fats, oils, and/or hydrophobic materials can be included in amounts of 1% to 10% w/w of the composition.

In some embodiments, the sensory perception of the texture modifying component is similar to that of fat, oil, or other hydrophobic materials. For example, a composition including 2.5% fats or oil can provide the same mouthfeel perception as a composition including 10%-50% fat as measured by sensory evaluation techniques.

Suitable oils and fats usable in compositions include vegetable or animal fats, such as butter, coconut oil, palm kernel oil, beef tallow, and lard, among others. These ingredients when used may be present in amounts up to about 7%, or up to about 3.5%, by weight of the composition.

In some embodiments, the composition may include edible oil component present in an amount of from about 1% to about 30%, alternatively 1% to 5%, alternatively 5% to 10%, alternatively 10% to 15%, alternatively 15% to 20%, alternatively 20% to 25%, alternatively 25% to 30%, by weight of the composition. In some embodiments, the edible oil component may be present in an amount of from about 0% to about 30%, alternatively 0% to 1%, alternatively 1% to 5%, alternatively 5% to 10%, alternatively 10% to 15%, alternatively 15% to 20%, alternatively 20% to 25%, alternatively 25% to 30%, by weight of the composition. This edible oil component makes up part of the group of ingredients that adjust the taste, texture, and improve the melt and mouth feel of the flavored chewy or gummy confection. For example, in some embodiments, the interaction of the group of highly unsaturated oils with the coconut oil component may create an improved elasticity within the flavored chewy confection that acts similar to hydrogenated or partially hydrogenated fat. The edible oil component also improves the health characteristic of the flavored chewy confection compositions because it adds monounsaturated and polyunsaturated fats. An example of an edible oil component is a blend of canola, soybean oil, and sunflower oil.

Non-limiting examples of edible oil components acceptable for use in the preferred embodiments include those that have low saturated fat content and high unsaturated fat including monounsaturated and especially polyunsaturated oils. The edible oil component should have no specific flavor and preferably is basically bland or somewhat buttery in taste. The edible oils component can be selected from the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof.

In some embodiment, the edible oil component may have a high level of saturated fats present in an amount of from about 0.3% to about 20%, alternatively 0.3% to 3%, alternatively 3% to 5%, alternatively 5% to 10%, alternatively 10% to 15%, alternatively 15% to 20%, by weight of the composition. Suitable oils having a high level of saturated fats include, but are not limited to, one or more selected from the group consisting of coconut oil, palm oil, fractionated coconut or palm oil, partially hydrogenated coconut or palm oil, fully hydrogenated coconut or palm oil, or any other synthesized or altered edible oils including partially hydrogenated oils and fully hydrogenated oils that have either highly saturated or highly unsaturated fatty acids that when hydrogenated become solid similar to coconut oil in consistency including partially hydrogenated soybean oil, cotton seed oil, palm kernel oil or combination of these edible oils. In one embodiment, the edible oil component comprises coconut oil. This oil component forms a part of the flavor profile and provides a texture to the flavored chewy or gummy confection, and it improves the taste, texture, melt, and mouth feel of the compositions. The blend of the flavor components provides for a great taste, texture, melt and mouthfeel, without the necessity of using partially hydrogenated or fully hydrogenated oils. Any medium heat processed coconut oil can be used.

Hemectant

The glycerin is a humectant and freezing point depressant. It also helps decrease the tendency for granulation and aid in maintaining softness. In some embodiment, glycerin or equivalent material may be employed at a level of from about 1 to about 5% by weight of the final product, e.g., 2 to 3%.

Humectants that can provide a perception of mouth hydration may be included. Such humectants can include, but are not limited to glycerol, sorbitol, polyethylene glycol, erythritol, and xylitol. Additionally, in some embodiments, fats can provide a perception of mouth moistening. Such fats can include medium chain triglycerides, vegetable oils, fish oils, mineral oils, and combinations thereof.

Bulking Agents

Suitable sugar bulking agents include monosaccharides, disaccharides and polysaccharides such as xylose, ribulose, glucose (dextrose), lactose, mannose, galactose, fructose (levulose), sucrose (sugar), maltose, invert sugar, partially hydrolyzed starch and corn syrup solids, and mixtures thereof.

Suitable sugar alcohol bulking agents include sorbitol, xylitol, mannitol, galactitol, lactitol, maltitol, erythritol, isomalt and mixtures thereof. Suitable hydrogenated starch hydrolysates include those disclosed in U.S. Pat. No. 4,279,931 and various hydrogenated glucose syrups and/or powders which contain sorbitol, maltitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN®, a commercially available product manufactured by Roquette Freres of France, and HYSTAR®, a commercially available product manufactured by SPI Polyols, Inc. of New Castle, Del., are also useful.

Emulsifiers

The composition may include an emulsifier. The emulsifier may present in an amount of from about 0.001% to about 5%, alternatively 0.001% to 1%, alternatively 1% to 3%, alternatively 3% to 5%, by weight of the composition. In some embodiments, the emulsifier present in an amount of from about 0% to about 5%, alternatively 0.001% to 1%, alternatively 1% to 3%, alternatively 3% to 5%, by weight of the composition.

Example emulsifiers include but not limited to modified corn starch, mono- and diglycerides, and lecithin.

The emulsifier may assist in holding together the fats and water and other components together in a homogeneous composition. In one embodiment, the emulsifier may assist in the formation of a “water and oil” emulsion that creates the smooth texture of the finished product.

Preservatives

Preservatives may be natural or synthetic. Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof. Natural preservatives may include phenols (phenolic acid, polyphenols, tannins), isoflavonoids, organic acids (acetic, lactic, citric), and herb extracts such as extracts of citrus fruits, oregano, thyme, sage, rosemary, clove, coriander, garlic, and onion.

In some embodiments, the composition may include at least about 0% to 2%, by weight of the composition of a preservative component from above, or mixtures thereof.

Liquids

Liquids may be used to assist in the flavoring and texture profile of the products. In some embodiments, the composition may include from about 0.001% to about 25% by weight of a fruit or vegetable or combination juice or concentrate component, alternatively 0.001% to 5%, alternatively 5% to 10%, alternatively 10% to 15%, alternatively 15% to 20%, alternatively 20% to 25%, by weight of the composition. The fruit or vegetable or combination juice or concentrate component adds a flavor to the flavored chewy or gummy confection. Any suitable source from the following may used in the embodiments; citrus fruit juices, orchard fruit juices, berry fruit juices, vine fruit juices, decolorized juices, and vegetable juices can be used for this component. The forms can come from juices or concentrates of fruits or vegetables.

In some embodiments, the composition may also include a water component present in an amount of about 0% or greater. The water component adds to the overall texture and melt and chewiness of the flavored chewy or gummy composition. For example, water may be used because of the increase in viscosity of some example compositions. In some embodiments, the composition may contain water from about 1% to about 20%, alternatively 1% to 5%, alternatively 5% to 10%, alternatively 10% to 15%, alternatively 15% to 20%, by weight of the composition.

Thickening Agent

The composition may further include a thickening agent to help with the viscosity of the final product. Some thickening agents are gelling agents. Others act as mechanical thixotropic additives with discrete particles adhering or interlocking to resist strain.

In some embodiments, the thickening agent may be polysaccharides or protein. Example polysaccharides thickening agents include starches, vegetable gums and pectin. Example starch based thickening agents include arrowroot, cornstarch, katakuri starch, potato starch, sago, tapioca and their starch derivatives. Example vegetable gums based thickening agents may include alginin, guar gum, locust bean gum, and xanthan gum. Example protein based thickening agents include collagen, egg whites, furcellaran, and gelatin. Sugar based thickening agent may include agar and carrageenan.

Gelling Components

The gelling compounds may include one or more gelling agents. A number of gelling agents may be utilized including without limitation, gelatin, pectin, gum Arabic, carrageenans, agar agar, high methoxy pectin, alginates, xanthan gum, locust bean gum, gellan gum, guar gum, modified or unmodified starches, cellulose gum, modified starch wheat flour or enriched wheat flour or bleached flour or any type of flour from a natural source, or a combination thereof. Other example gelling agents may include acacia, alginic acid, bentonite, Carbopols® (now known as carbomers), carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, and tragacanth.

The amount of gelling agents used in the composition depend upon the texture, viscosity and softness of a desired product as well as other ingredients in the composition In some embodiments, to gelling agents may be used in concentrations of about 0.5% to about 10%, about 0.1% to about 7%, or about 0.2 to about 15%.

In one embodiment, the gelatin and pectin may be employed at a weight ratio supplying at least 50% gelatin and at least 10% pectin, e.g., from about 70 to 85% gelatin and the remainder pectin.

In one embodiment, pectin may be a high methoxy pectin obtained from apples. In one embodiment, gelatin may be a type A gelatin from porcine sources. Bloom values for the gelatin may be in the range of from 100 to 280. In one embodiment, the bloom value is about 250.

In one embodiment, the combination of gelatin and pectin may be employed at a level of from about 4.5 to about 6% by weight of the final product, e.g., about 5.5% on that basis.

In one embodiment, the composition may include gellan gum, carrageenans, or both providing a gelatin free composition. In one embodiment, the composition may include from about 0.25% to about 0.75% by weight gellan gum and about 2% to about 3% by weight carrageenan based on the total weight of the composition.

In one embodiment, a combination of gellan gum at about 0.25% to about 0.75% by weight and carrageenans at about 2.5% to about 3% by weight based on total weight of the product produces a gummy composition with TPA hardness values in excess of 20 lbs(f), and TPA cohesiveness and elasticity values of 75% to 80%.

In one embodiment, the amount of gellan gum is about 0.25 wt % to about 0.75 wt %, and about 0.25 wt % to about 0.5 wt %. In one embodiment, the amount of carrageenan is about 1.5 wt % to about 3 wt %, and about 2.5 wt % to about 3 wt %.

Texture Profile Analysis (TPA)

Texture Profile Analysis (TPA) is used to characterize the aspects of the texture of a gelled or gummy product. Specifically, TPA cohesiveness is a measure of the resiliency of a product, TPA elasticity is a measure of the springiness, and TPA hardness is a measure of firmness. For example, a gelatin gel tends to have high resiliency and high springiness, along with relatively high firmness.

Gelatin-based gummy composition may have both TPA cohesiveness and TPA elasticity values in the range of 80% to 90%, and TPA hardness values in the range of 25 to 30 lbs(f). Gelatin alternatives commonly found in the marketplace, such as starch and pectin, typically attain the proper TPA hardness, but have lower TPA elasticity values ranging from 30% to 50% and much lower TPA cohesiveness values ranging from 15% to 30%. Gellan gum has produced products with acceptable TPA hardness, TPA elasticity in the 50% to 70% range and TPA cohesiveness in the range of 30% to 50%. Carrageenan as a gelatin alternative have produced products with TPA cohesiveness and elasticity values of 75% and higher, but with unacceptably low TPA hardness values in the 10 to 15 lbs(f) range.

The following are typical TPA analysis of a several gelatin-based gummy products:

| | TPA | TPA | TPA |
|---|---|---|---|
| Product | Hardness lbs(f) | elasticity % | cohesiveness % |
| A | 30 +− 3 | 86 +− 1 | 82 +− 3 |
| B | 25 +− 2 | 91 +− 1 | 86 + 3 |
| C | 28 +− 2 | 90 +− 2 | 81 +− 1 |
| D | 28 +− 3 | 89 +− 3 | 83 +− 3 |

Nutraceutical Product Kit

In another aspect, the application provides a nutraceutical product kit. The kit may include several products packaged together

In one embodiment, the nutraceutical product kit may include a first product, comprising a neuroprotecting composition, a second product, comprising an anti-inflammatory composition, a third product, comprising a cardiovascular protecting composition, and a fourth product, comprising an antioxidant composition.

In one embodiment, the nutraceutical product kit may further include a fifth product, comprising a telomerase composition. In one embodiment, the nutraceutical product kit may further include a sixth product, comprising a vitamin composition. In one embodiment, the nutraceutical product kit may further include a seventh product, comprising a mineral composition.

Each product in the kit may be in a form selected from gummy, gel, tablet, capsule, soft-chew, powder, or a combination thereof. In one embodiment, multiple gummy products may be packaged in one box or pouch to provide a daily package. In one embodiment, multiple tablets may be packaged in one box or pouch to provide a daily package.

Methods of Making

Processing typically starts with the preparation of a boiled mixture of mixed sugars which is then blended with the gelling component and processed into shapes by depositing into starch moulds. It can also be simply cast onto a slab or cast into rubber moulds. The pieces are then held to set and dry. For a general description of this type of process, see Lees and Jackson; Sugar Confectionery and Chocolate Manufacture; 1973 (ISBN 0249 44120 9); pages 226-268. This reference is incorporated by reference herein in its entirety.

The method may further include a final processing process in which the final product is prepared. This process can include, for example, extruding, thermoforming, molding, shaping, cutting, and the like, to form the final product in the desired shape. Those skilled in the art are capable of designing a suitable final processing procedure to prepare the final gelled or gummy products, depending on the desired texture (e.g., chewy or gummy) and shape (e.g., cube, square, sheet, animal shaped, etc.).

The embodiments now will be described in more detail with reference to the non-limiting examples that follow.

### EXAMPLES

The following examples are provided to illustrate the preparation of gummy confections in accordance with this invention and are provided by way of illustration and are not intended to limit the invention in any way.

### Example 1: A Nutraceutical Gummy Composition

Ingredients: Sucrose, Maltose, Maltodextrin, Sorbitol, Glucose Syrup, L-leucine, L-isoleucine, L-valine, Water, Potassium Sorbate, Sodium Benzoate, Gelatin, nutraceutical premix, and Candy Apple Green Flavor

The nutraceutical premix include:
A neuroprotecting agent including Melatonin, Acetyl L-carnitine, Alpha-lipoic acid, or Ginseng (Siberian) extract;
A anti-inflammatory agent including Acetylsalicylic acid, Curcumin, or Ginger root extract;
A cardiovascular protecting agent including DHA and EPA; and
A antioxidant agent including Bioflavonoids, Pomegranate extract, Ginkgo biloba, Green tea extract, Garlic extract, Resveratrol, CoEnzyme Q10, Vitamin C, Vitamin E, Quercetin, Rutin, Lycopene, L-glutathione (GSH), or N-acetyl cysteine.

The gelatin, sorbitol, nutraceutical premix, potassium sorbate, and sodium benzoate were shifted together until homogenous using a fork. The dry component mixture was added to water rapidly with rapid stirring. The dry mix quickly absorbed the water and became a bright yellow rubbery mass. The rubbery mass was added to a container and heated at 160° F. until free of foam and a clear yellow.

In a separate container were combined water, glucose syrup, maltose, maltodextrin and brought to a boil. The sucrose was added. The solution was brought to 200° F. and the gelatin solution was slowly added with stirring. The mixture was stirred until homogenous. The flavoring was then added. The solution was then added to silicone molds and the molds were placed in the refrigerator for 90 minutes.

### Example 2: A Nutraceutical Product Kit

A nutraceutical product kit including gummy products with the ingredient listed as following:
A neuroprotecting gummy including Melatonin, Acetyl L-carnitine, Alpha-lipoic acid, or Ginseng (Siberian) extract;
A anti-inflammatory gummy including Acetylsalicylic acid, Curcumin, Ginger root extract;
A cardiovascular protecting gummy including DHA and EPA;
A antioxidant gummy including Bioflavonoids, Pomegranate extract, Ginkgo biloba, Green tea extract, Garlic extract, Resveratrol, CoEnzyme Q10, Vitamin C, Vitamin E, Quercetin, Rutin, Lycopene, L-glutathione (GSH), or N-acetyl cysteine;
A vitamin gummy including Vitamin B1, Vitamin B3, Vitamin B6, Vitamin B12, Folic acid, Vitamin C, Vitamin D, Vitamin E, and Vitamin A;
A mineral gummy including potassium, chromium pocolinate, magnesium, selenium, and Zinc; or
A telomerase activating gummy including astragalus extract.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A nutraceutical composition, comprising,
a neuroprotecting composition,
an anti-inflammatory composition,
a cardiovascular protecting composition,
an antioxidant composition, and
a gelling composition in a sufficient amount to provide a cohesive gelled product.

2. The nutraceutical composition of claim 1, further comprising a telomerase activating composition, a mitochondria boosting composition, a neuro-stimulant composition, a vitamin composition, a mineral composition, or an amino acid composition.

3. The nutraceutical composition of claim 1, wherein the neuroprotecting composition consists of melatonin, acetyl L-carnitine, alpha-lipoic acid, ginseng, its extract or isolates, or a combination thereof.

4. The nutraceutical composition of claim 1, wherein the anti-inflammatory composition comprises acetylsalicylic acid, bioflavonoids, omega-3 fatty acids, white willow, curcumin, turmeric, saffron, ginger, boswallia, chamomile, licorice, arnica, witch hazel, Echinacea, bromelain from pineapple, yarrow, oak bark, walnut leaf, fenugreek, aloe vera, calendula, capsaicin, mallow, wild pansy, extract thereof, powder thereof, or a combination thereof.

5. The nutraceutical composition of claim 1, wherein the cardiovascular protecting composition comprises DHA, EPA, ALA, omega-3 fatty acids, cod liver oil, fish oil, algae oil, flax seed oil, or a combination thereof.

6. The nutraceutical composition of claim 1, wherein the antioxidant composition comprises bioflavonoids, pomegranate, Ginkgo biloba, green tea, garlic, grapeseed, vitamin E, vitamin C, resveratrol, coenzyme Q10, quercetin, rutin, lycopene, L-glutathione, N-acetyl cysteine, phenolics, anthocyanins, flavonoids, anthracenes, carotenoids, zeaxanthin, astaxanthin, blackberry, elderberry, cranberry, blueberry, saffron, Sangre de grado (dragon's blood), Lyceum barbarum (Gouqizi), their extract, power, or derivative thereof.

7. The nutraceutical composition of claim 2, wherein the telomerase activating composition comprises Astragalus, its extract or isolates, astragaloside, astragenol, cycloastragenol, or derivatives thereof.

8. The nutraceutical composition of claim 2, wherein the mitochondria boosting composition comprises acetyl L-carnitine, alpha-lipoic acid, coenzyme Q10 (CoQ10, or ubiquinone), Shilajit extract or powder, nicotinamide riboside (NR), vitamin B, vitamin D, omega-3 fatty acids, magnesium, D-ribose, or a derivative or combination thereof.

9. The nutraceutical composition of claim 2, wherein the neurostimulant composition comprises caffeine or an herbal extract comprising caffeine or its derivative thereof.

10. The nutraceutical composition of claim 2, wherein the amino acid composition comprises at least one essential amino acid or its derivative thereof.

11. The nutraceutical composition of claim 2, wherein the vitamin composition comprises vitamin A, B, C, D, E, K or a combination thereof.

12. The nutraceutical composition of claim 11, wherein the vitamin composition comprises Vitamin B1 (thiamine), Vitamin B2 (riboflavin), niacin (B3), vitamin B6, vitamin B12, folic acid, pantothenic acid, biotin, or derivatives thereof.

13. (canceled)

14. The nutraceutical composition of claim 1, wherein the gelling composition comprises pectin and gelatin in a ratio from about 10:1 to about 1:1.

15. The nutraceutical composition of claim 1, wherein the gelling composition comprises hydrolyzed collagen.

16. The nutraceutical composition of claim 1, further comprising an additive selected from sweeteners, food acids, flavoring agents, coloring agents, humectants, bulking agents, fatty acids, triglycerides, plasticizers, emulsifiers, thickeners, preservatives, or and a mixture thereof.

17. The nutraceutical composition of claim 16, wherein the sweetener comprises erythritol, xylitol, sugar, glucose syrup, corn syrup, high fructose corn syrup, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

18. The nutraceutical composition of claim 16, wherein the food acid comprises malic acid, fumaric acid, lactic acid, tartaric acid, glucono-delta lactone, salts of gluconic acid, phosphoric acid, succinic acid, adipic acid, acetic acid, citric acid, or a combination thereof.

19. (canceled)

20. (canceled)

21. (canceled)

22. A nutraceutical product kit, comprising,
a first product, comprising a neuroprotecting composition,
a second product, comprising an anti-inflammatory composition,
a third product, comprising a cardiovascular protecting composition, and
a fourth product, comprising an antioxidant composition.

23. The nutraceutical product kit of claim 22, further comprising a fifth product comprising a telomerase composition, a sixth product comprising a vitamin composition, or a seventh product comprising a mineral composition.

24. The nutraceutical product kit of claim 22, wherein each product is in a form selected from gummy, gel, tablet, capsule, soft-chew, powder, or a combination thereof.

25. (canceled)
